(19) United States
(12) Patent Application Publication
(10) Pub. No.: US 2023/0234059 A1
(43) Pub. Date: Jul. 27, 2023

(54) SYSTEM AND METHOD FOR PARTICLE SIZE-INSENSITIVE HIGH-THROUGHPUT SINGLE-STREAM PARTICLE FOCUSING

(71) Applicant: The University of Hong Kong, Hong Kong (CN)

(72) Inventors: Kevin Kin Man Tsia, Hong Kong (CN); Chak Man Lee, Hong Kong (CN)

(21) Appl. No.: 18/151,107

(22) Filed: Jan. 6, 2023

Related U.S. Application Data
(60) Provisional application No. 63/266,543, filed on Jan. 7, 2022.

(51) Int. Cl. B01L 3/00; G01N 21/01

(52) U.S. Cl. CPC B01L 3/502761; B01L 3/502707; B01L 3/502715; G01N 21/01; B01L 2200/10; B01L 2200/0652; B01L 2300/0809; B01L 2300/0861; B01L 2400/0487; G01N 2021/1765

(57) ABSTRACT

A tunable inertial sheathing (TIS) system and methods for particle-size-insensitive high-throughput single-stream focusing of particles suspended in a particle-carrying fluid are provided. The TIS conditions particles to distribute locally within one of compartments of inertial force field, followed by an inertial focusing to migrate it to a single foci. For the particle localization, the TIS system introduces an arbitrary form of peripheral sheathing by generating and accumulating sheath fluid from particle-carrying fluid through a combination of inertial focusing, channel bifurcation and channel confluence. Multiple forms of the TIS system are also provided, each including one main channel and at least one bypass channel. The main channel includes and cascades at least three segments, at least one bifurcating junction and at least one confluence junction.

### CROSS-REFERENCE TO RELATED APPLICATION

This application claims the benefit of U.S. Provisional Application Serial No. 63/266,543, filed Jan. 7, 2022, which is hereby incorporated by reference in its entirety including any tables, figures, or drawings.

### BACKGROUND OF THE INVENTION

Focusing particles into a single stream in a high-speed fluid flow provides high precision, efficiency, and throughput for particle analysis, sorting, counting and filtering. The operation of high-throughput single-stream focusing is crucial for a wide range of applications such as flow cytometry or single-cell sorting. The conventional approaches in this field employ either external force field, for example, electromagnetic force field or acoustic force field, or sheath/particle-free fluid for hydrodynamic focusing, to actively confine the positions of the particles.

However, the manufacturing costs of these conventional systems aiming to achieve optimal focusing performance are generally prohibitively high, owing to the requirements of precise control of the force field and the fluid flow rate. Moreover, when the hydrodynamic focusing mechanism is used, the large amount of sheath fluid significantly dilutes the particle concentrations, making further downstream particle analysis difficult to be scalable in throughput.

Inertial focusing is a proven focusing method that can bypass expensive force-field controlling devices and sheath fluid. The way it aligns particles, which induces a converging force field (known as inertial force field) purely by a high-speed fluid flow in a microchannel, is promising in lowing the manufacturing and operation costs. Despite its passiveness, high-throughput and high precision, the only fly in the ointment is that it does not naturally form single stream. The naturally compartmental inertial force field, where one foci per compartment, aligns particles that are uniformly distributed into multiple particle streams.

Achieving single-stream inertial focusing thus necessities a reduction of number of particle streams. State-of-the-art approaches can be categorized into single-field (Cat1) and multi-field (Cat2) where the former can be sub-categorized into single-staged (Cat11) and multi-staged (Cat12). Single-staged single-field and multi-field approaches engineer a single-foci force field that aligns particles into single stream at once. On the other hand, multi-staged single-field approaches engineer a sequence of multi-foci inertial force field that aligns particles progressively from multiple streams to single stream. In other words, these approaches rely on the use of multiple force fields but are different in the way of implementation, i.e., in parallel (Cat12 and Cat2) and in sequence (Cat12). Notable approaches in each category are: (Cat1) straight pipe with non-rectangular cross-section, (Cat12) straight pipe with varying cross-sections, (Cat2) curved pipe with a rectangular cross-section, (Cat2) straight rectangular pipe with a periodically varying aspect ratio and (Cat2) straight rectangular pipe with non-Newtonian fluid.

These diverse approaches yet share one critical disadvantage that is the tradeoff between the throughput (for example, flow rate) and the particle size tolerance. It can be understood from the finite coverage of effective flow rate and particle size of each force field. The use of multiple force fields results in a narrower effective coverage. For instance, high-throughput approaches only effectively focus monodisperse particles (e.g., size variation less than 10 µm) and the maximum throughput of high-tolerance approaches is generally below 10,000 particle per second. These two quantities are >3 times and > 10 times lower than that of commercially available hydrodynamic focusing, respectively. It is insufficient for real-life scenarios that involve large-scale polydisperse particles (i.e., particles that have large size variation) such as flow cytometry, where typical biological cell sizes span from 5 to 30 micrometers or more with a concentration > le5 per mL of sample. Furthermore, it is challenging for the existing single-stream inertial-focusing approaches to achieve high practicability due to design tradeoff among various factors such as precision, flow rate tolerance, and manufacturing-friendliness.

### BRIEF SUMMARY OF THE INVENTION

There continues to be a need in the art for improved designs and techniques for a passive, cost-effective and high-performance single-stream particle focusing system and methods for particle analysis and filtering on a microscale.

Embodiments of the subject invention pertain to a particle focusing system and methods for particle-size-insensitive high-throughput single-stream inertial focusing, for example, polydisperse particles are aligned into a single stream without requiring multiple internal force field or any external force fields or external sheath fluid. The method of the subject invention is based on localizing the particle distribution on channel cross-section to accommodate the compartmental nature of inertial force field. In the absence of external force field, a particle distribution that localizes within only one compartment of the inertial force field leads to the formation of a single stream. This method avoids perturbing the inertial force field, thereby maximizing the effective coverage of fluid flow rate and particle size to meet the practical needs. The particle localization is achieved in the system of tunable inertial sheathing (TIS), which can generate an arbitrary pattern of peripheral sheathing using the inertial force. In other words, TIS condenses particles to a narrower stream with an arbitrary shape and position on the channel cross-sectional like the conventional hydrodynamic focusing while using the inertial force instead of sheath fluid. Uniquely, it accumulates the inertial wall-effect to turn a physics-defined sheathing into an arbitrary sheathing. The accumulation comprises of two iterative processes: (1) induce the inertial wall-effect to peripherally sheath particles and (2) physically partition the channel periphery to isolate the sheath fluid from particles and simultaneously further induce wall-effect to particles. In the language of physics, this iteration continuously stores the work-done by the abstract wall-effect into a potential energy in form of actual sheath fluid. Upon sufficient accumulation, removing all partitions unleashes the accumulated wall-effect (sheath fluid) that instantaneously localizes particle distribution on the channel cross-section.

According to an embodiment of the subject invention, a TIS system for particle localization is provided, which comprises a main channel and at least one bypass channel for accumulating and depleting the inertial wall-effect (equivalently sheath fluid in the context of TIS). All bypass channels carry the sheath fluid and each comprises at least one inlet and at least one outlet. The main channel carries the particle-carrying fluid and comprises at least three straight segments; at least one bifurcation junction; and at least one confluence junction. Straight segments are joint by the bifurcation and confluence junctions disposed in between, which connects to the inlet and outlet of bypass channel, respectively. From the sheathing perspective, a TIS system can be decomposed into 4 types of functional blocks. The long straight segment in the very beginning, which initiates the inertial focusing to peripherally sheath particles, serves as an initiation unit (block A). A bifurcation junction with a long straight segment attached in the end, where the bifurcation junction divides certain previously generated sheath fluid to the bypass channel and the straight segment with sufficiently long length recovers the sheathing, constructs an accumulation unit (block B). A confluence junction with a short straight segment attached in the end, where the confluence junction returns certain sheath fluid to the main channel to temporally localize particles in the straight segment, constructs a depletion unit (block C). A bypass channel, in which only sheath fluid flows, serves as a storing unit (block D).

The main channel of TIS system comprises of one block A in the beginning, at least one block C at the downstream, and at least one block B in between to enable particle localization. The tunability of TIS system can be enhanced by using a greater number of block B and C to form a complex structure, which can be characterized by the arrangement of block B and block C into 3 classes: interleaved (i.e., BCBC...BCBC), blocked (i.e., BB...BBCC...CC), and by part (i.e., mixture of interleaved and blocked). Note that the abovementioned structure enables TIS in only one direction (one-way TIS). A TIS system that enables an arbitrary particle localization on channel cross-section would comprises at least four one-way TIS systems for a sufficient degree-of-freedom on two dimensions.

In certain embodiments of the subject invention, a one-way TIS system comprises a microchannel comprising a main channel having a high-aspect-ratio rectangular cross-section for single-stream focusing on a planar channel design. The main channel, a high-aspect-ratio rectangular pipe, is formed for continuous inertial focusing and its inertial force field is configured to halve the cross-sectional area of the channel into two horizontally parallel compartments. The one-way TIS systems firstly focus a uniformly distributed particles by the inertial force into focus of the two compartments, wherein the foci is an equilibrium point of inertial force field located apart from long walls of the channel and at the center of the long wall, to form two particles streams. It then progressively increases the inertial sheathing one-sidedly to posit two streams into the same compartment, which allows merging them into single stream by sole inertial focusing.

In a certain embodiment of the subject invention, the one-way TIS system, which is tailored for simplifying channel design process, comprises an interleaved form comprising a block A and 4 pairs of block B and block C, each pair connects to a block D with a progressively decreased hydraulic resistance. In another embodiment of the of the subject invention, the one-way TIS system, which is tailored for biological applications, comprises a blocked form comprising a block A, 6 block B and 1 block C, all connect to the same block D. In these embodiments, a bifurcating junction is first formed and is configured for dividing the sheath fluid from the main channel to the bypass channel. It physically partitions the channel cross-section such that a thin slide of sheath fluid between one of the two particle streams and its nearest wall of the channel is precisely isolated from the main channel. Particularly, this fluid slide generated by the inertial wall-effect is not tunable and has a thickness depending on Reynolds number, sizes of the particles, and geometry of the channel. This partition results in a sheath fluid in the branch; a main channel with two particle streams where one of it relatively shifts towards the wall. A segment of main channel follows and is configured to have the similar inertial force field to refocus two particle streams to the focus, thereby generate an extra particle-free slide. In the interleaved embodiment, a confluence junction is then formed and configured to sheath the particle-carrying fluid one-sidedly, thereby temporally localize two particle streams into a smaller compartment skewed toward one side. The process is then iterated for 3 more times that progressively increase the amount of extracted sheath fluid. Eventually, the amount of sheath fluid in the bypass channel is equal to or more than the particle-carrying fluid in the main channel (equivalently occupies one compartment of the inertial force field), which localizes two particle streams into a compartment of the inertial force field. On the other hand, in the blocked embodiment, the extraction process is iterated for 5 more times to complete the sheath accumulation before returning sheath fluid back to the main channel. Additionally, all bifurcation junctions associate with a well at bifurcation point to avoid its direct contact with particles, thereby reducing the impact on particles and promote particle viability. After the localization, a long straight channel is formed the same as or similar to the previous straight segment to continue inertial focusing, which merges two localized particle streams into a single stream.

### BRIEF DESCRIPTION OF THE DRAWINGS

FIG. 1 is a schematic representation of a microfluidic chip with annotation defining geometrical terms used herein, according to an embodiment of the subject invention.

FIG. 2A is a schematic representation illustrating the compartmental nature of inertial force field. Inertial force fields comprise of many repulsive areas that join into lines. These lines deny particle crossing and cut through the whole field to act as imaginary partitions, which divide the force field and results a compartmental structure. Due to this nature, particle only migrate to the foci corresponding to the compartment it initially stays.

FIG. 2B is a schematic representation illustrating particle stream reduction enabled by a localized particle distribution in the beginning of inertial focusing. Single-stream focusing can be achieved by inertial focusing with a localized particle distribution that is confined within a compartment of the inertial force field, according to an embodiment of the subject invention. It is different compared with inertial focusing with a uniform particle distribution in the number of resultant particle streams.

FIG. 3A shows schematic representation of a passive sheathing at the periphery of a channel cross-section with a square shape induced by the wall-induced lift force during inertial focusing.

FIG. 3B shows schematic representations of peripheral sheathing by inertial focusing in different shapes of the cross-sections of the channels. All cross-sections of the channels give a peripheral sheathing regardless of the exact pattern of their inertial force field.

FIG. 3C is a schematic representation showing the principle of the tunable inertial sheathing (TIS) process in a cross-sectional view. TIS focuses on the particle distribution on the channel boundary, from which inertial focusing, specifically its wall-effect induced lift force, repels particles. This repulsion is equivalent to passively sheath the particle-carrying fluid. A channel bifurcation then forms a physical partition to divide this portion of particle-free fluid to a bypass channel. It simultaneously stores the wall-effect in form of sheath fluid and resets the particle distribution in the original channel. Another inertial focusing follows to induce additional wall-effect to particles on the original channel and thus another sheathing. At last, a channel confluence joins two separate channels together to form a thickened sheath that results a more localized particle distribution away from the boundary. An iteration of these steps enables a passive while tunable sheathing.

FIG. 4A is a schematic representation showing the principle of arbitrary particle localization by TIS process. A complete TIS system comprises of at least four one-way TIS system, where each compresses the particle distribution in one direction through adjusting the peripheral inertial sheathing, to give sufficient degree-of-freedom for localizing particles to a distribution with an arbitrary size and position on a channel cross-section. In each one-way TIS, it comprises at least one a sequence of bifurcation (bi), inertial focusing (In) and confluence (Con) connected in series to form an interleave structure for accumulating sufficient sheath fluid.

FIG. 4B is a schematic representation of an alternative design of one-way TIS system comprising a sequence of bifurcation (bi) and inertial focusing (In) at the upstream and a confluence (Con) at the downstream to form a blocked form.

FIG. 5 is a schematic representation of single-stream focusing by TIS in rectangular cross-sections with different aspect ratio. An extreme aspect ratio reduces the complexity of compartmentation of inertial force field from 2D partition to a 1D partition, thereby reduces complexity of TIS system for single stream focusing.

FIG. 6A is a schematic representation of microfluidic pattern designs for TIS system, according to an embodiment of the subject invention. The panel titled TIS system shows the 3D view and the top view of a TIS system, which comprises one main channel and one bypass channel, wherein the main channel having a high-aspect-ratio rectangular cross-section comprises a bifurcation junction and a confluence junction connected sandwiched by three straight segments, and the bypass channel consists of a segment of arbitrary shape. This system can be decomposed into 4 building blocks (i.e., block A, B, C, and D) for different controls of sheathing, which are detailed on the right panels at a top view.

FIG. 6B is a schematic representation illustrating the particle trajectories in a pressure-driven flow within the TIS system at a top view. Cross-sectional views at six representative locations show the corresponding cross-sectional migration. Particles first migrate into two streams and are then biased towards one of the two compartments via TIS, according to an embodiment of the subject invention. Conditioned in this localized particle distribution, a subsequent inertial focusing results in a single stream.

FIG. 6C shows a schematic representation of equivalent electric circuit model of the TIS system with a basic form and illustrates the change of velocity flow profile before and after development and its relationship with the width and volumetric flow rate of particle-containing and particle-free fluids.

FIG. 7A shows schematic representations of inertial focusing effect of particles with two different sizes in channel having a high-aspect-ratio rectangular cross-section and its effect on single stream formation using different TIS approaches, according to an embodiment of the subject invention.

FIGS. 7B-7C are schematic representations of different embodiments of the TIS system, wherein FIG. 7BA is a schematic representation of the TIS system having an interleaved form, FIG. 7C is a schematic representation of the TIS system having a blocked form, according to an embodiment of the subject invention. For each embodiment, its geometry, an exploded diagram showing the composition of building blocks, and an equivalent electric circuit model of the bifurcating structure for analysis, are given according to an embodiment of the subject invention.

FIG. 8 shows a simulation of peripheral sheathing extraction process by TIS system. The result is shown by a series of streamline plot where the portion of fluid designed to be branch out to the bypass is in orange color. By varying the width of the bypass channel, the fluid volume divided to the bypass is shown to be controlled by the hydraulic resistance ratio between the bypass and the bridge. Moreover, the bifurcation takes only the peripheral fluid that is at the bypass’s side - giving a well-defined partitioning effect to the channel cross-section, according to an embodiment of the subject invention.

FIG. 9A shows a simulation of particle flow trajectory in a TIS system with the basic structure. The bottom particle stream shows a temporal shift right after the channel confluence, which supports a particle localization given by the TIS system, according to an embodiment of the subject invention.

FIG. 9B shows an in-scale schematic representation of the TIS system with the basic structure wherein the zoom-in window shows shapes of a bifurcating junction and a confluence junction of the TIS system indicated by gray boxes, according to an embodiment of the subject invention.

FIG. 9C shows two pairs of fluorescent streak images respectively illustrating flow trajectories of a solution carrying fluorescent polystyrene beads of a diameter of 8 µm and a solution carrying fluorescent polystyrene beads of a diameter of 20 µm passing the bifurcating junction and the confluence junction of a TIS system with the basic form, showing a clear stream shift after the confluence junction, according to an embodiment of the subject invention

FIG. 9D shows ultrafast laser scanning images of a stream of fast-flowing biological cells (1) before reaching the bifurcating junction, (2) after passing the bifurcating junction, and (3) after passing the confluence junction, wherein the red arrows indicate gaps between the biological cell stream and the adjacent channel wall and the yellow dotted lines indicate the center of the main channel, according to an embodiment of the subject invention.

FIG. 10A shows fluorescent streak images illustrating the difference of particle flow trajectories in the TIS system comprising a bifurcation junction with and without an expanded well, proving that an expanded well effectively avoid particle collisions, which does not affect the particle localization after the confluence but could deform or damage the cells, and FIG. 10B shows cell images captured by ultrafast laser scanning microscope before and after the particle collisions, wherein the triangular-shaped cells, which are significantly deformed cells indicated by the red arrows, are found only after the particle collisions, according to an embodiment of the subject invention.

FIG. 11A shows a simulation of particle flow trajectory in a TIS system with the interleaved form. The bottom particle stream shows a temporal shift right after every channel confluence with an increasing amplitude, which supports a progressive accumulation of inertial sheath that localizes particle to one of the compartments of inertial focusing and leads to a single-stream focusing, according to an embodiment of the subject invention.

FIG. 11B is an in-scale schematic representation of microfluidic pattern design having the TIS system with the interleaved form for particle localization, wherein the zoom-in window shows the bifurcating junctions and the confluence junctions indicated by the gray boxes, and FIG. 11C shows seven pairs of fluorescent streak images illustrating the flow trajectories of 8 µm and 20 µm fluorescent polystyrene beads at all the junctions, according to an embodiment of the subject invention.

FIG. 12A shows the co-flow of the water and ink in a TIS system with the block structure. By increasing the water-to-ink ratio, the flow pattern shows that the bypass channel progressively intakes the peripheral fluid in the main channel and returns all stored fluid back to the main channel, according to an embodiment of the subject invention.

FIG. 12B is an in-scale schematic representation of microfluidic pattern design having the TIS system with the blocked form for particle localization, and FIG. 12C shows four fluorescent streak images illustrating the flow trajectories of 6 µm fluorescent polystyrene beads across the entire TIS system and the subsequent long straight segment, according to an embodiment of the subject invention.

FIG. 13 shows laser scanning images of tests evaluating the TIS assisted inertial focusing performance with the use of a TIS system when microparticles and cultured cells flowing through the microfluidic channel at three different flow rates, wherein the laser scanning images are captured both before the TIS system (equivalently sole inertial focusing) and after the TIS system to evaluate the particle-size-insensitive single-stream focusing performance, according to an embodiment of the subject invention.

FIG. 14A shows a compressed image containing 10,000 cells taken within 1 second and FIG. 14B shows images of the first 120 cells of FIG. 14A for demonstrating the performance of the TIS assisted inertial focusing on heterogeneous cell samples at high throughput, according to an embodiment of the subject invention.

### DETAILED DISCLOSURE OF THE INVENTION

Embodiments of the subject invention are directed to a tunable inertial sheathing (TIS) system and methods for particle-size-insensitive high-throughput single-stream inertial focusing of particles suspended in a particle-carrying fluid.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not prelude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e., the value can be +/- 10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

### Working Principle of Particle Localization for Size-Insensitive Single-Stream Inertial Focusing

Various embodiments of the TIS system and methods described below are based upon the notion of a microfluidic channel design that is capable of focusing particles with a broad size distribution into a single stream of a high-speed, for example, Newtonian, microfluidic flow. The TIS system and method enable high precision, efficiency and throughput for particle detection, analysis and filtering on a microscale. Thus, the systems and method of the subject invention are suitable for applications in various fields of microfluidic, analytical chemistry, cell biology, clinical diagnostic, health care, marine and life science research.

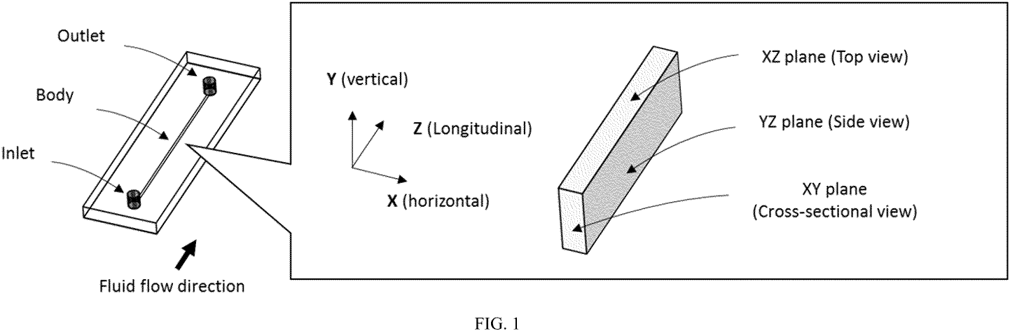

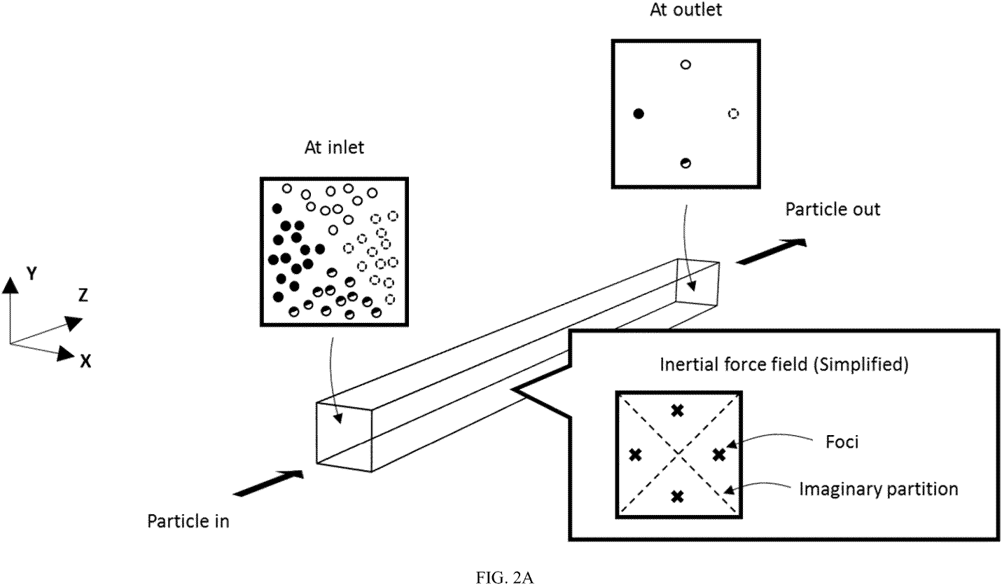

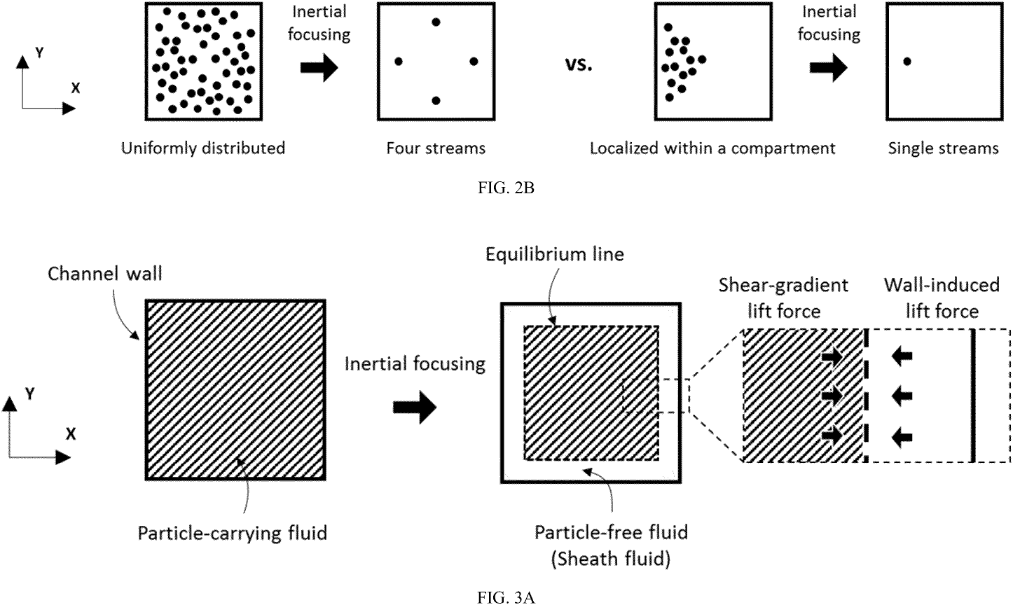

The TIS system and methods of the subject invention are designed to assist the inertial focusing for single-stream particle focusing. It is noted that the term “particle focusing” used herein refers to confining positions of particles in the cross-section of the channel by cross-streamline migration of the particles. The terms, cross-section, and other geometrical terms describing the microfluidic pattern are illustrated in FIG. 1. In the inertial focusing technology solutions, the particle focusing is introduced by two counteracting inertial lift forces, namely, shear-gradient-induced lift force and wall-induced lift force, which act toward and away from the wall of the channel, respectively. Referring to FIG. 2A, such force pair forms an inertial force field at the cross-section of the channel, which naturally forms a plurality of compartments, each compartment having a repulsive boundary, which denies particles from crossing, and a foci where the net force is zero. The particles automatically migrate to the focal spot corresponding the compartment it locates on the cross-section during its travelling down the stream when Reynolds number of the flow is sufficiently large, the traveling distance is sufficiently long and particle size is similar to the hydraulic diameter of the channel cross-section. The Reynolds number (Re) and the hydraulic diameter (Dₕ) are defined by following equation:

$$R e = \frac{ρ ⋅ {U}_{m} ⋅ {D}_{h}}{μ} a n d {D}_{h} = \frac{4 A}{P}$$

where
ρ = carrier fluid densityUₘ = maximum flow velocityµ = dynamic viscosity of the fluidDₕ = hydrlic diameter of channel = 4A/P A = area of the channel cross sectionP = perimeter of the channel cross sectionDₕ In certain embodiments of the invention, Re is between 1 and 2000 and is between 100 nm and 100 mm for enabling inertial focusing. Referring to FIG. 2B, due to the compartmental nature of inertial force field, the number of particle streams being formed is also governed by the particle distribution in the cross section of the channel. A plurality of particle stream would be formed by sole inertial focusing under a normal condition where particles are uniformly distributed. Reversely, one can obtain a single stream purely by inertial focusing with a particle distribution that localizes within single compartments - the central idea of the subject invention.
ρ = carrier fluid densityUₘ = maximum flow velocityµ = dynamic viscosity of the fluidDₕ = hydrlic diameter of channel = 4A/P A = area of the channel cross sectionP = perimeter of the channel cross section Dₕ
ρ = carrier fluid densityUₘ = maximum flow velocityµ = dynamic viscosity of the fluidDₕ = hydrlic diameter of channel = 4A/P A = area of the channel cross sectionP = perimeter of the channel cross section Dₕ
ρ = carrier fluid densityUₘ = maximum flow velocityµ = dynamic viscosity of the fluidDₕ = hydrlic diameter of channel = 4A/P A = area of the channel cross sectionP = perimeter of the channel cross section Dₕ
ρ = carrier fluid densityUₘ = maximum flow velocityµ = dynamic viscosity of the fluidDₕ = hydrlic diameter of channel = 4A/P A = area of the channel cross sectionP = perimeter of the channel cross section Dₕ
ρ = carrier fluid densityUₘ = maximum flow velocityµ = dynamic viscosity of the fluidDₕ = hydrlic diameter of channel = 4A/P A = area of the channel cross sectionP = perimeter of the channel cross section Dₕ
ρ = carrier fluid densityUₘ = maximum flow velocityµ = dynamic viscosity of the fluidDₕ = hydrlic diameter of channel = 4A/P A = area of the channel cross sectionP = perimeter of the channel cross section Dₕ

The advantage of single-stream focusing by particle localization is the expansion of effective particle size, which fulfill the needs of application that involves particles with a broad size distribution, e.g., particle focusing for flow cytometry and particle filtration. Comparing to the existing single-stream inertial focusing approaches, this approach can be done without a significant force field modification to bypass its associated size-dispersion effect. A notable example is using Dean flow, which introduces an additional Dean force (F_{D}) having a second-order dependency on particle sizes as illustrated by the force ratio (R_{f}).

$${R}_{f} = \frac{{F}_{L}}{{F}_{D}} = \frac{2 ⋅ R ⋅ {a}^{2}}{{D}_{h}}$$

where
F_{L} = Inertial lift forceF_{D} = Dean forceR = Radius of curvaturea = particle sizeDₕ = hydralic diameter
F_{L} = Inertial lift forceF_{D} = Dean forceR = Radius of curvaturea = particle sizeDₕ = hydralic diameter
F_{L} = Inertial lift forceF_{D} = Dean forceR = Radius of curvaturea = particle sizeDₕ = hydralic diameter
F_{L} = Inertial lift forceF_{D} = Dean forceR = Radius of curvaturea = particle sizeDₕ = hydralic diameter
F_{L} = Inertial lift forceF_{D} = Dean forceR = Radius of curvaturea = particle sizeDₕ = hydralic diameter

Such force naturally distributes particles of difference sizes across the channel cross-section and results in a plurality of particle stream, each formed by particles with a specific size. In other words, the definition of single-stream focusing of the existing approaches is applicable only to certain particles that have a specific size. This phenomenon inevitably limits the conventional approaches to size-based particle separation and detours them from the single-stream particle focusing. Thus, a particle localization that avoids perturbing the inertial force field is the key to expand the effective coverage of fluid flow rate and particle size of single-stream inertial focusing.

### The System and Methods of Tunable Inertial Sheathing (TIS) for Particle Localization

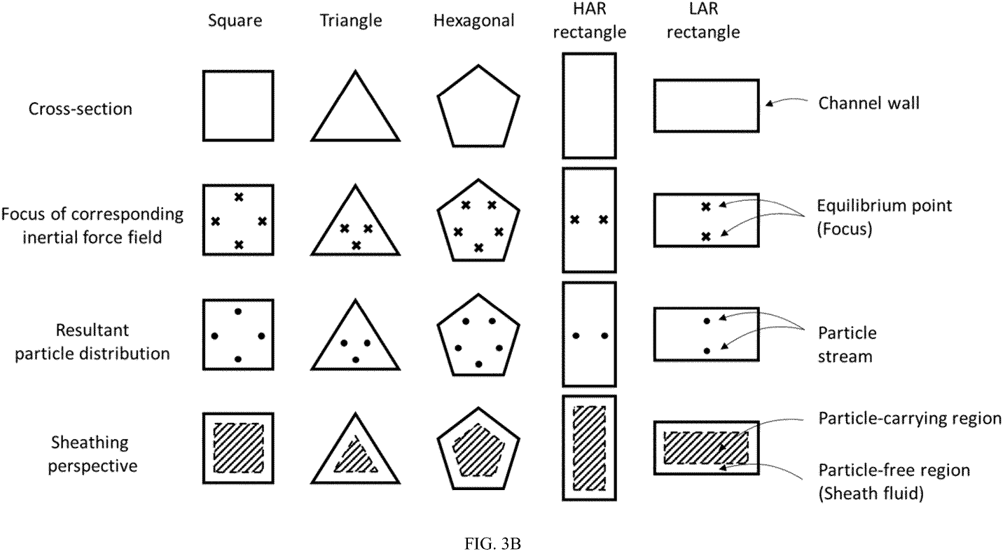

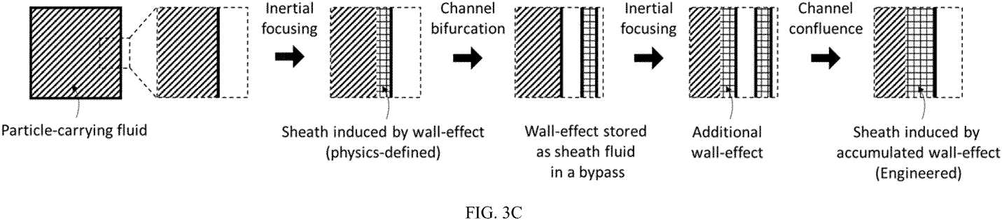

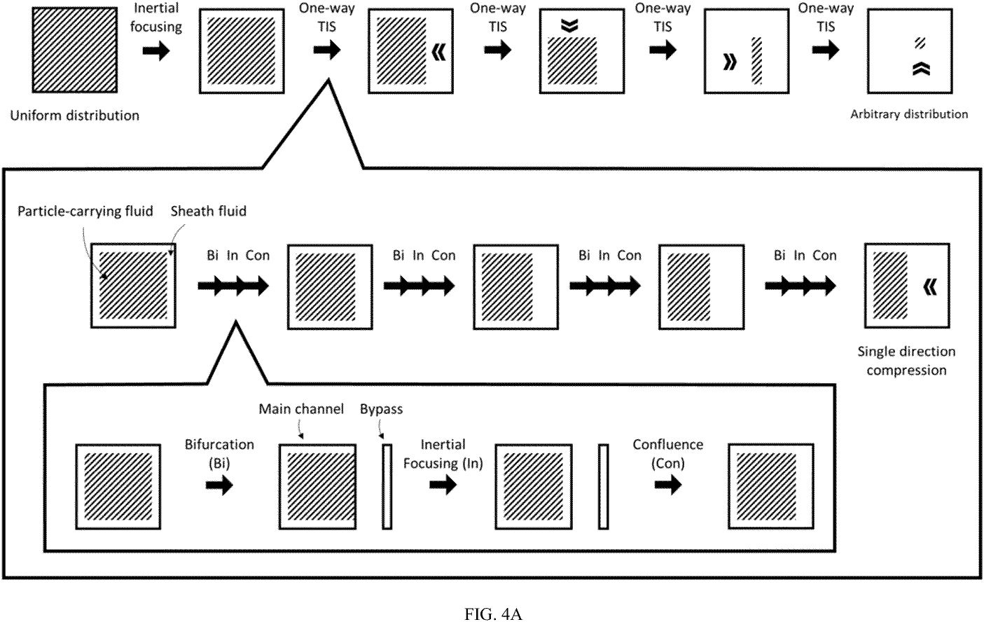

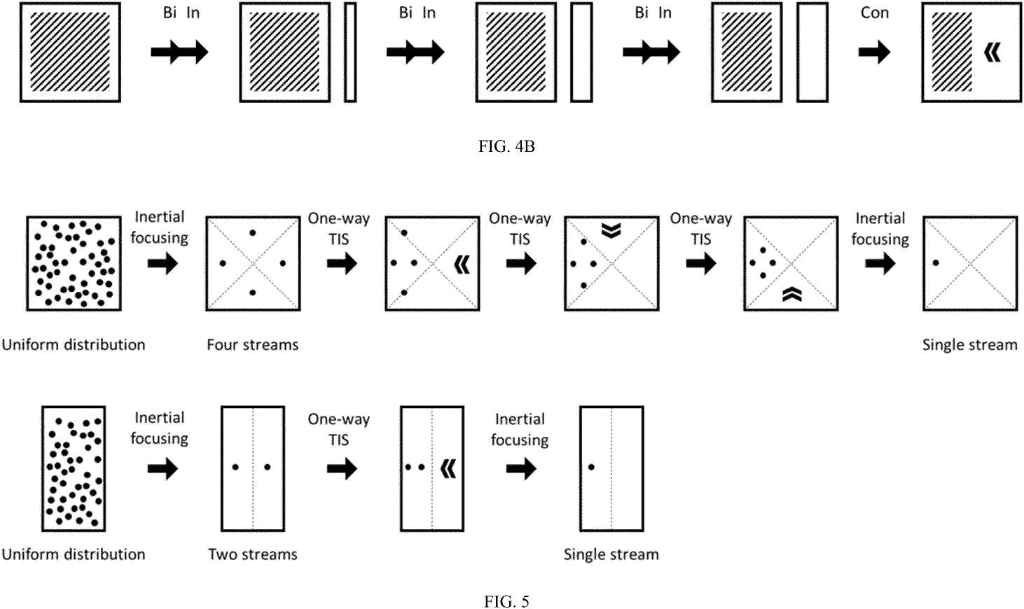

TIS system and methods of the subject invention achieves arbitrary particle localization on a channel cross-section without any force field modification through engineering the peripherally sheathing induced by the inertia wall effect, which effectively shapes the envelope of the particle distribution. Referring to FIG. 3A, one of the signature particle migrations by inertial focusing is the off-the-wall movement, which is equivalent to a peripheral sheathing, induced by the wall-induced life force. Referring to FIG. 3B, the fact that all foci of inertial force field must sit on the equilibrium line makes the concept of peripheral sheathing generic to any cross-sectional shape of the channel regardless of the exact form of inertial force. Note that the thickness of this peripheral sheath is not arbitrary but defined by Re, flow profile and the size of particle being focused. Referring to FIG. 3C, TIS system engineers the thickness through sheath accumulation through at least one cycle of channel bifurcation, inertial focusing and channel confluence. In detail, a channel bifurcation forms a physical partition to divide certain sheath fluid to a bypass channel. This action simultaneously stores the wall-effect in form of sheath fluid and resets the particle distribution in the original channel. A following inertial focusing induces wall-effect again to repel particles on the original channel and thus additional sheathing. Then, a channel confluence joins two separate channels together to form a thickened sheath. Referring to FIG. 4A, a one-way TIS system of subject invention enables an iteration of this sheath accumulation cycle, which flexibility adjusts the thickness of sheath along one direction. An arbitrary particle distribution can thus be delivered by at least four one-way TIS systems. Referring to FIG. 4B, a one-way TIS system can be alternatively formed by at least one cycle of channel bifurcation and inertial focusing and a channel confluence at the end. In general, the fewer the number of compartments of the inertial force field, the fewer the one-way TIS system is needed for single-compartment localization, thereby more practical. Referring to FIG. 5, for instance, at least three one-way TIS system is required to localize particles into single compartment of inertial force in a channel with a square cross-section. In practice, a channel with a high-aspect-ratio rectangular cross-section is employed to result only two compartments on the inertial force field, which simplifies designs and manufacturing of TIS system, where the aspect ratio (AR) defined as

$$A R = \frac{H}{W}$$

$$A R = \frac{H}{W}$$

where
H = the channel height
W = the channel width

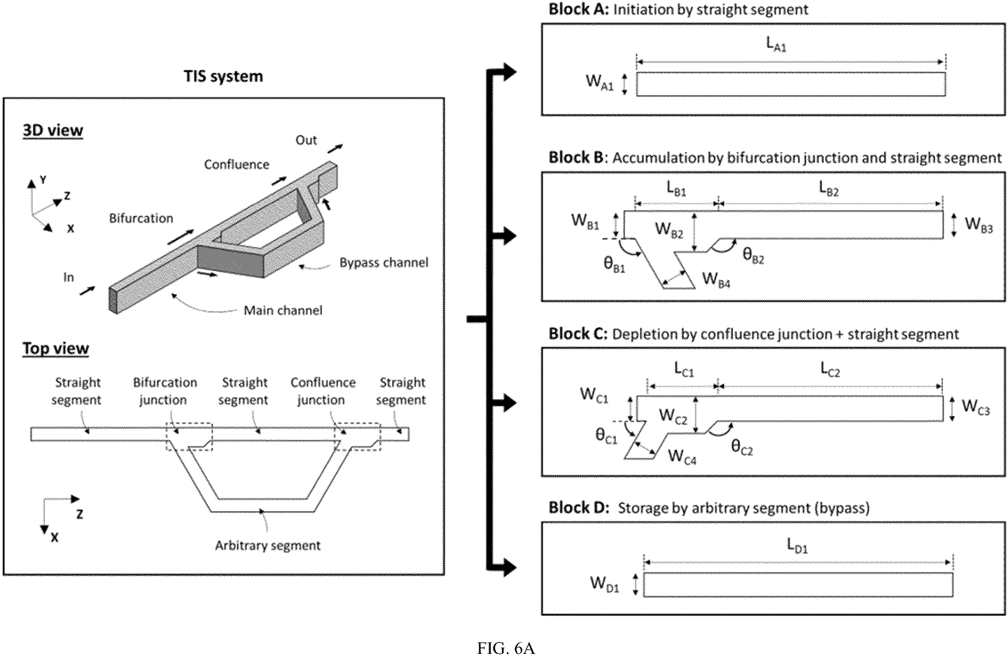

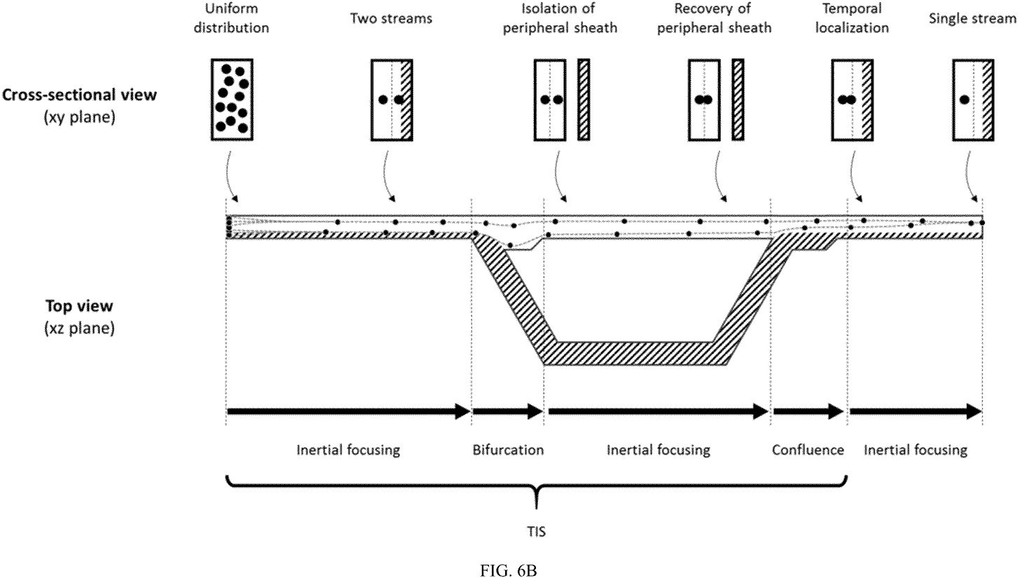

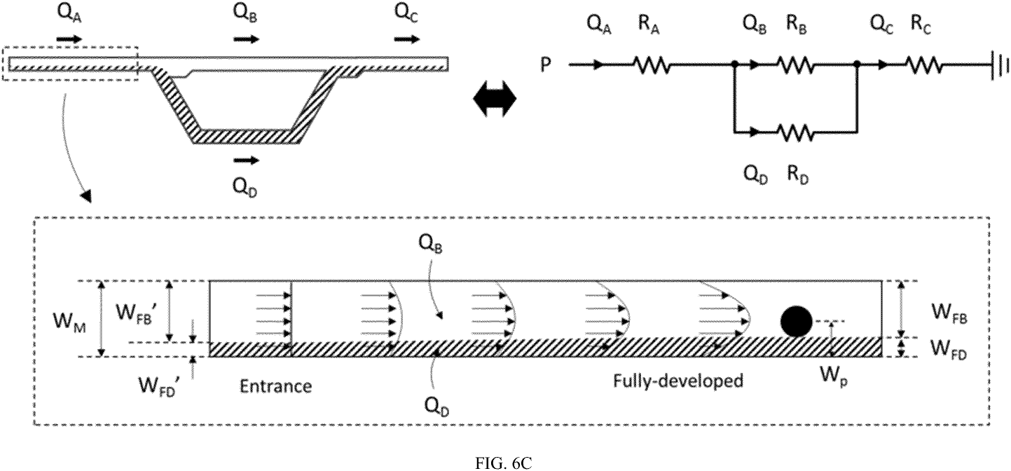

In certain embodiments of the invention, the height H and the width W each is between 1 micrometer and 10 millimeter and an aspect ratio AR is between 0 and 0.75 or larger than 1.33 for partitioning the inertial force field into two compartments. Referring to FIG. 6A, the TIS system of the subject invention can be implemented by a properly designed and constructed pattern, which comprises a main channel and a bypass channel. The main channel has a high-aspect-ratio rectangular cross section and comprises at least one bifurcating junction, at least one confluence junction and at least three straight segments and the bypass channel comprises an arbitrary segment. The shape of each segment can be configured based on different design criteria. Typically, every straight segment has an extended cuboid for continuous inertial focusing and may have a structure other than a cuboid for other design purposes, for example, channel expansion or contraction for inter-particle spacing control and slight bending for flexible design, as long as there is no significant secondary flow being introduced into the straight segments. A bifurcation junction has one inlet and two outlets connected to the two straight segments and a bypass channel, respectively. A confluence junction has two inlets and one outlet connected to the two straight segments and one bypass channel, respectively. Both types of junctions may have an expanded well attached downstream. In each junction, the angle between two straight segments may vary between -90 degrees and 90 degrees and the angle between the straight segment at the inlet and the bypass channel may vary between 0 degree and 180 degrees.

The TIS system can be decomposed into four building blocks, each responsible for different functions. The very first straight segment of the main channel is block A for initializing inertial focusing; a bifurcation junction of the main channel with a long straight segment attached at one end is block B for accumulating sheath fluid in the bypass channel; a confluence junction of the main channel with a straight segment attached at one end is block C for depleting sheath fluid from the bypass channel; and an arbitrary segment of bypass channel is block D for storing sheath fluid.

In an embodiment of the invention, the fluidic channel of TIS comprises silicone, for example, but not limited to polydimethylsiloxane.

In an alternative embodiment of the invention, the fluidic channel of CPC comprises cyclic olefin copolymer (COC), polymethylmethacrylate (PMMA), or polycarbonate (PC).

In one embodiment illustrated by FIG. 6A, the simplest functional form, which is a basic form, of the TIS system comprises a main channel assisted with a unifying structure.

Referring to FIG. 6B, in the TIS system with the basic form, the first straight segment provides an inertial focusing to continuously drag particles to one of the two foci on the mid-plane surrounded by peripheral sheath fluid. A bifurcation junction divides a well-defined portion of sheath fluid into the bypass channel at one side and leaves a pair of skewed particle streams in the main channel. The particle streams are repositioned to the focal spots by inertial focusing to reform the sheath before reaching the confluence junction. At the confluence junction, the sheath fluid previously stored in the bypass merges with the particle-carrying fluid in the main channel to further sheath the particles away from the partition side that results in a temporal particle localization. Given sufficient propagation downstream, the inertial force field merges the particle streams into single stream if two streams are located at the same compartment.

To form and maintain particles streams on the mid-plane by inertial focusing, the average cross-sectional areas of every straight segment of the TIS system is configured to have a high aspect ratio. Accordingly, a width W_{A1} of block A, and a width W_{B1}, a width W_{B2} and a width W_{B3} of block B, and a width W_{C1}, a width W_{C2} and a width W_{C3} of block C, are configured to satisfy the condition below:

$${W}_{n} \leq \frac{H}{A R}$$

where
n = A1, B1, B2, B3, C1, C2 and C3
Wₙ = channel width of segment nH = channel heightAR = aspect ratio ≥ 1.33
Wₙ = channel width of segment nH = channel heightAR = aspect ratio ≥ 1.33
Wₙ = channel width of segment nH = channel heightAR = aspect ratio ≥ 1.33

In certain embodiments of subject invention, the height H and the width W each is between 1 micrometer and 10 millimeters and an aspect ratio AR is between 1.33 and 4 for dividing the cross-sectional area of the channel into two horizontally parallel compartments and thus a pair of streams on the mid-plane.

In one embodiments of subject invention, the height H is about 80 micrometers, the width W is between 20 and 60 millimeters, and the aspect ratio AR is between 1.33 and 4.

In addition, a theoretical range of the focusable size can be determined by the minimum channel width to satisfy the condition below:

$$0.07 H \leq {D}_{p} < min \left({W}_{n}\right)$$

Where
n = A1, B1, B2, B3, C1, C2 and C3Dₚ = particle sizeWₙ = channel width of segment nH = channel height
n = A1, B1, B2, B3, C1, C2 and C3Dₚ = particle sizeWₙ = channel width of segment nH = channel height
n = A1, B1, B2, B3, C1, C2 and C3Dₚ = particle sizeWₙ = channel width of segment nH = channel height
n = A1, B1, B2, B3, C1, C2 and C3Dₚ = particle sizeWₙ = channel width of segment nH = channel height

In certain embodiments of the subject invention, Dₚ is between 0.1 micrometers and 10 millimeters.

In one embodiments of the subject invention, Dₚ is between 5.6 and 30 micrometers.

Further, a length L_{A1} of block A and a length L_{B2} of block B are configured to satisfy the condition below in order to achieve the inertial focusing:

$$\begin{matrix}{L}_{n} \geq \frac{3 π ⋅ μ ⋅ {D}_{n}^{2}}{4 ⋅ ρ ⋅ {U}_{m} ⋅ {a}^{3}} \left(\frac{{W}_{n}}{{C}_{L}^{-}} + {\left(\frac{H}{{C}_{L}^{+}}\right)}^{k}\right) , H > {W}_{n} , {C}_{L}^{+} < < {C}_{L}^{-} , \\ k = \left\{\begin{matrix}1 , n = A 1 \\ 0 , n = B 2\end{matrix}\right)\end{matrix}$$

where
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient
n = A1 and B2 ρ = carrier fluid densityµ = dynamic viscosity of the fluidDₙ
= hydrlic diameter of segment nUₘ = maximum flow velocitya
= minimum particle sizeH = channel heightW
= channel width of segment
$$n {C}_{L}^{-}$$
= negative lift coefficient
$${C}_{L}^{+}$$
= positive lift coefficient

It is noted that particles migrate along both the vertical axis (y-axis) and the horizontal axis (x-axis) of the straight segment of block A from random positions to foci, while the particles migrate mostly along the horizontal axis (x-axis) once focused (for example, in block B). Accordingly, the length L_{A1} of block A is configured to be equal to or greater than the length L_{B2} of block B.

In one embodiment, the length L_{A1} of block A is configured to be on a scale of tens of millimeters, while the length L_{B2} of the block B is configured to be on a scale of a few millimeters.

On the other hand, it is desirable that the particle migration in the bifurcation junction of block B and in block C is minimized. Thus, a length L_{B1} of block B and a length L_{C1} and a length L_{C2} of block C are configured to be much shorter than the length L_{A1} of block A or the length L_{B2} of block B.

In one embodiment, a length L_{B1} of block B and a length L_{C1} and a length L_{C2} of block C each is configured to be on a scale of hundreds of micrometers.

Referring to FIG. 6C, the bifurcating junction is configured to perform fluid bifurcation that divides sheath fluid to the bypass channel. At the entrance, the fluid flow is related to the stream width by the following equation:

$${Q}_{A} = {W}_{M} ⋅ H ⋅ v$$

$${Q}_{B} = {{W}^{′}}_{F B} ⋅ H ⋅ v$$

$${Q}_{D} = {W}_{F D} {}^{′} ⋅ H ⋅ v$$

where
Qₙ = volumetric flow rate in block n, where n = A, B and DWₙ
   = channel width of segment nH = channel heighv
   = linear fluid flow rateW_{FB}′
   = width of entrance stream to the block BW_{FD}′
   = width of entrance stream to the block D
Qₙ = volumetric flow rate in block n, where n = A, B and DWₙ
   = channel width of segment nH = channel heighv
   = linear fluid flow rateW_{FB}′
   = width of entrance stream to the block BW_{FD}′
   = width of entrance stream to the block D
Qₙ = volumetric flow rate in block n, where n = A, B and DWₙ
   = channel width of segment nH = channel heighv
   = linear fluid flow rateW_{FB}′
   = width of entrance stream to the block BW_{FD}′
   = width of entrance stream to the block D
Qₙ = volumetric flow rate in block n, where n = A, B and DWₙ
   = channel width of segment nH = channel heighv
   = linear fluid flow rateW_{FB}′
   = width of entrance stream to the block BW_{FD}′
   = width of entrance stream to the block D
Qₙ = volumetric flow rate in block n, where n = A, B and DWₙ
   = channel width of segment nH = channel heighv
   = linear fluid flow rateW_{FB}′
   = width of entrance stream to the block BW_{FD}′
   = width of entrance stream to the block D
Qₙ = volumetric flow rate in block n, where n = A, B and DWₙ
   = channel width of segment nH = channel heighv
   = linear fluid flow rateW_{FB}′
   = width of entrance stream to the block BW_{FD}′
   = width of entrance stream to the block D

To ensure fluid in bypass channel is particle-free, the width of fully developed stream to the branch cannot exceed that of the particle-free fluid slice in a straight segment, which is fully determined by the Reynolds number Re of the fluid, the particles sizes, and the channel geometry. Referring to FIG. 6C again, taking the development of flow velocity profile into account, following condition is obtained:

$${W}_{P} \geq {W}_{F D} ⇒ {W}_{P} \geq {f}_{p a r a} \left({W}_{F D} {}^{′}\right)$$

where
W_{P} = Distance from the particle to the nearest channel wallW_{FD}
= width of fully developed stream to the block
$$D {W}_{F D}^{′}$$
= width of entrance stream to the blockDƒₚₐᵣₐ
= mapping function between entrance and fully developed flow
W_{P} = Distance from the particle to the nearest channel wallW_{FD}
= width of fully developed stream to the block
$$D {W}_{F D}^{′}$$
= width of entrance stream to the blockDƒₚₐᵣₐ
= mapping function between entrance and fully developed flow
W_{P} = Distance from the particle to the nearest channel wallW_{FD}
= width of fully developed stream to the block
$$D {W}_{F D}^{′}$$
= width of entrance stream to the blockDƒₚₐᵣₐ
= mapping function between entrance and fully developed flow
W_{P} = Distance from the particle to the nearest channel wallW_{FD}
= width of fully developed stream to the block
$$D {{W}^{′}}_{F D}$$
= width of entrance stream to the blockDƒₚₐᵣₐ
= mapping function between entrance and fully developed flow

In certain embodiment of subject invention, a width of fully developed stream to the block D, W_{FD}, is configured to be between 0 to 60 micrometers.

In a pressure-driven microfluidic flow, the volumetric flow rate is governed by following equation:

$$Q = \frac{Δ P}{R} ∝ \frac{1}{{R}_{H}}$$

where
Q = volumetric flow rateΔP = pressure differnt between inlet and outletR = hydralic resistance
Q = volumetric flow rateΔP = pressure differnt between inlet and outletR = hydralic resistance
Q = volumetric flow rateΔP = pressure differnt between inlet and outletR = hydralic resistance

Therefore, to control the bifurcation, it is important to control the hydraulic resistance. For a high-aspect-ratio cuboid channel, the hydraulic resistance of the channel is approximately governed by following condition:

$$R ∝ \frac{L}{{W}^{2}}$$

where
R_{H} = hydralic pressureL = channel lengthW = channel width
R_{H} = hydralic pressureL = channel lengthW = channel width
R_{H} = hydralic pressureL = channel lengthW = channel width

As a result, the sheath extraction can be obtained by properly configuring the ratio of hydraulic resistance and thus the ratio of the width and the length between the block B and the block D as shown by the equation below:

$$\frac{{W}_{F B}}{{W}_{F D}} = \frac{{f}_{p a r a} \left({W}_{F B} {}^{′}\right)}{{f}_{p a r a} \left({W}_{F D} {}^{′}\right)} = \frac{{W}_{B 3}^{2} ⋅ {L}_{D 1}}{{W}_{D 1}^{2} ⋅ {L}_{B 2}}$$

where
W_{FB} = width of fully developed stream to the block BW_{FD} = width of fully developed stream to the block
$$D {{W}^{′}}_{F B}$$
= width of undeveloped stream to the exit
$$s e g m e n {{W}^{′}}_{F D}$$
= width of undeveloped stream to the branchƒₚₐᵣₐ
= mapping function between undeveloped and fully developed flowWₙ
= channel width of segment nLₙ = length of the segment n
W_{FB} = width of fully developed stream to the block BW_{FD}
= width of fully developed stream to the block
$$D {{W}^{′}}_{F B}$$
= width of undeveloped stream to the exit
$$s e g m e n {{W}^{′}}_{F D}$$
= width of undeveloped stream to the branchƒₚₐᵣₐ
= mapping function between undeveloped and fully developed flowWₙ
= channel width of segment nLₙ = length of the segment n
W_{FB} = width of fully developed stream to the block BW_{FD}
= width of fully developed stream to the block
$$D {{W}^{′}}_{F B}$$
= width of undeveloped stream to the exit
$$s e g m e n {{W}^{′}}_{F D}$$
= width of undeveloped stream to the branchƒₚₐᵣₐ
= mapping function between undeveloped and fully developed flowWₙ
= channel width of segment nLₙ = length of the segment n
W_{FB} = width of fully developed stream to the block BW_{FD}
= width of fully developed stream to the block
$$D {{W}^{′}}_{F B}$$
= width of undeveloped stream to the exit
$$s e g m e n {{W}^{′}}_{F D}$$
= width of undeveloped stream to the branchƒₚₐᵣₐ
= mapping function between undeveloped and fully developed flowWₙ
= channel width of segment nLₙ = length of the segment n
W_{FB} = width of fully developed stream to the block BW_{FD}
= width of fully developed stream to the block
$$D {{W}^{′}}_{F B}$$
= width of undeveloped stream to the exit
$$s e g m e n {{W}^{′}}_{F D}$$
= width of undeveloped stream to the branchƒₚₐᵣₐ
= mapping function between undeveloped and fully developed flowWₙ
= channel width of segment nLₙ = length of the segment n
W_{FB} = width of fully developed stream to the block BW_{FD}
= width of fully developed stream to the block
$$D {{W}^{′}}_{F B}$$
= width of undeveloped stream to the exit
$$s e g m e n {{W}^{′}}_{F D}$$
= width of undeveloped stream to the branchƒₚₐᵣₐ
= mapping function between undeveloped and fully developed flowWₙ
= channel width of segment nLₙ = length of the segment n
W_{FB} = width of fully developed stream to the block BW_{FD}
= width of fully developed stream to the block
$$D {{W}^{′}}_{F B}$$
= width of undeveloped stream to the exit
$$s e g m e n {{W}^{′}}_{F D}$$
= width of undeveloped stream to the branchƒₚₐᵣₐ
= mapping function between undeveloped and fully developed flowWₙ
= channel width of segment nLₙ = length of the segment n

To facilitate the analysis of the complex hydraulic resistance of the pressure-driven microfluidic pattern of the TIS system with a basic form, an analysis of an equivalent electric circuit model that is analogous to the TIS system with a basic form as shown in FIG. 6C can be employed.

Note that if the extracted sheath layer has a thickness approximate to the gap between a particle and the channel wall, a bifurcation leads to particle collision with the bifurcation point. An expanded well attached at the end of the bifurcation junction would avoid this strong impact to particle and thus promote the viability of the particles, which is essential in applications involving live biological cells. Similarly, a well attached at the end of the confluence junction also inhibits the particle collision under strong influence of inertia. Moreover, the transition between the expanded well to the subsequent straight segment should not be blunt to inhibit generating strong secondary flow. In certain embodiments of subject invention, a width W_{B2} and a width W_{C2} each is between 1 micrometer and 10 millimeter and an angle θ_{B2} and an angle θ_{C2} each is between 120 degrees and 180 degrees. In one embodiment of subject invention, a width W_{B2} and a width

W_{C2} each is between 40 micrometers and 100 millimeters and an angle θ_{B2} and an angle θ_{C2} each is between 120 degrees and 170 degrees.

Given that the final amount of sheath fluid is the sum of all generated sheath fluid in a TIS system, a sufficient condition to localize all particles within one compartment for single stream focusing is defined as:

$$\left(1 + 1\right) {W}_{p} \geq \frac{{W}_{m}}{2} ⇒ 4 {W}_{p} \geq {W}_{m}$$

where
Wₘ = channel width of block AW_{P}
   = Distance from the particle to the nearest channel wall
Wₘ = channel width of block AW_{P}
   = Distance from the particle to the nearest channel wall
Wₘ = channel width of block AW_{P}
   = Distance from the particle to the nearest channel wall

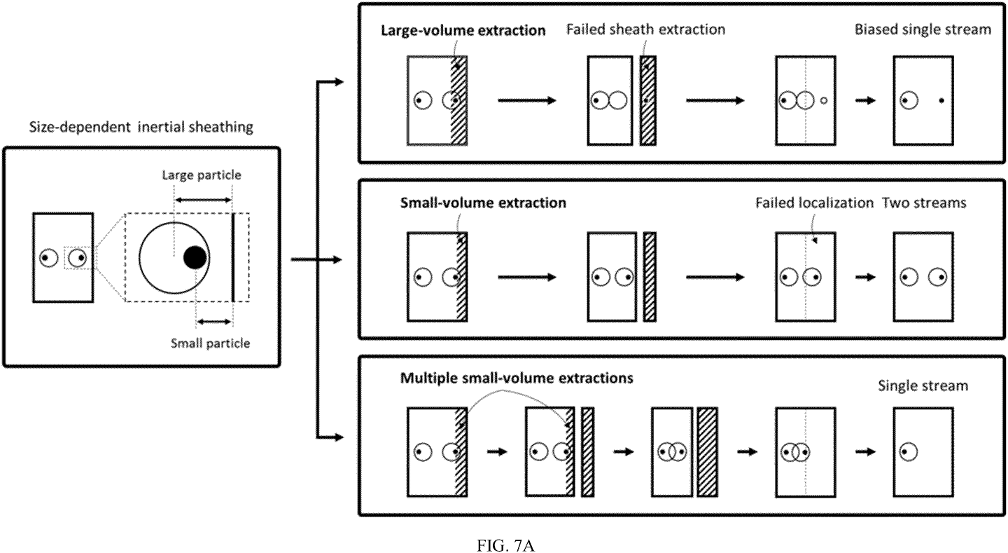

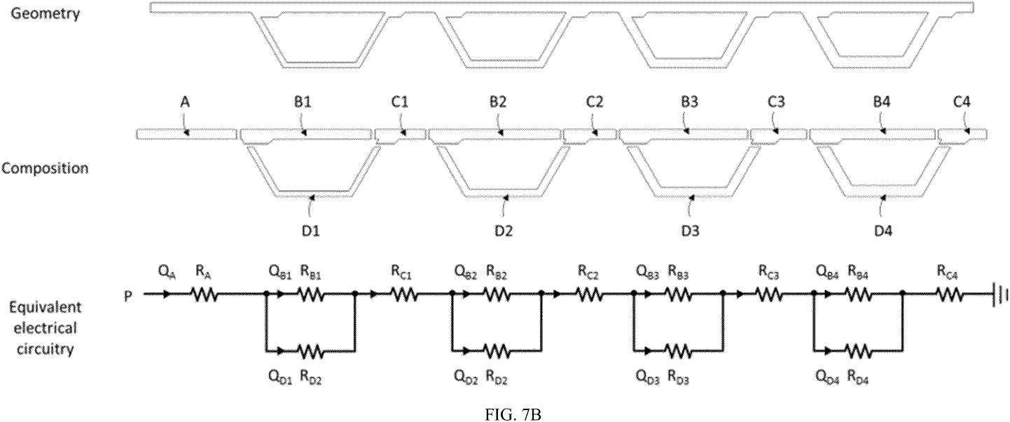

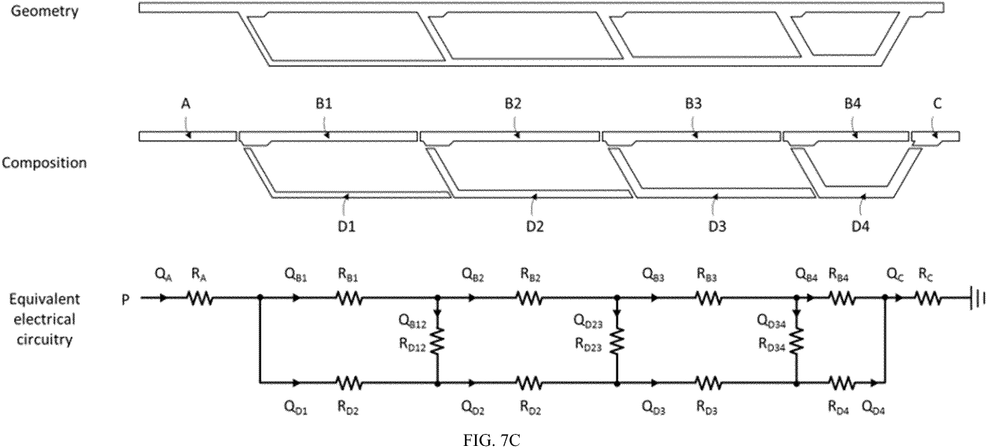

Despite that the sheath fluid is tunable in the TIS system with a basic form, the volume of the sheath fluid generally may not be sufficient to bias all particles into one compartment of the original channel’s cross-sectional area after one sheath accumulation cycle of TIS. Referring to FIG. 7A, two particles with different sizes are now inertially focused in a channel having a rectangular cross section with a high aspect ratio. Besides all particles are focused on the midplane, there is a size-dependent particles sheathing by the inertial wall-effect along the horizontal direction. Specifically, the larger the particle, the further it moves away from the channel wall. To satisfy the single-stream particle localization condition at the single sheath accumulation cycle, the fluid being branched out must be sufficiently large and inherently cover the small particles, resulting in failed sheath extraction and thus a size-dependent single stream focusing. On the other hand, if one extracts a small volume, it is generally not sufficient to satisfy the single-compartment localization condition. As a result, a pair of symmetrical focal points still remains.

In order to achieve size-insensitive single-stream focusing, equivalently broad effective particle size coverage, one must resort to a multi-cycle TIS as illustrated in FIG. 7A to accumulate sufficient volume of sheath fluid without branching out small particles. It can be achieved by cascading multiple building blocks to construct a complex form of TIS system, including but not limited to an interleaved form, a blocked form, or a mixed form by combining one or more of these previously mentioned building blocks.

In one embodiment, as illustrated in FIG. 7B, the TIS system has the interleaved form that includes a plurality of block B and block C connected to each other to form a periodic cascade having loops of bifurcation and confluence, each pair of block B and block C connecting to a block D. The resistance ratio between block B and block D is progressively decreasing down the stream to monotonically increase the sheath amount stored in the bypass channel.

In another embodiment, as illustrated in FIG. 7C, the TIS system has the blocked form that includes a plurality of block B integrated into one structure with a block C in the end that only a single Block D with multi-inlets is formed. In comparison with the interleaved form, the design of the blocked form offers advantages of a smaller footprint and a lower total resistance, though requiring more complex channel analysis.

The different forms of the TIS system can be combined in various manners, creating more sophisticated architecture for TIS. For example, in one embodiment, a mixed form comprising a random combination of building blocks can be constructed for a microfluidic network in a laminar flow.

Same set of design rules in TIS system can be applied to these complex forms. A sufficient condition to localize all particles within one compartment for single stream focusing now becomes:

$$\left(1 + n\right) {W}_{p} \geq \frac{{W}_{m}}{2} ⇒ 2 \left(1 + n\right) {W}_{p} \geq {W}_{m}$$

where
Wₘ = channel width of block AW_{P}
   = Distance from the particle to the nearest channel walln = number of block B
Wₘ = channel width of block AW_{P}
   = Distance from the particle to the nearest channel walln = number of block B
Wₘ = channel width of block AW_{P}
   = Distance from the particle to the nearest channel walln = number of block B

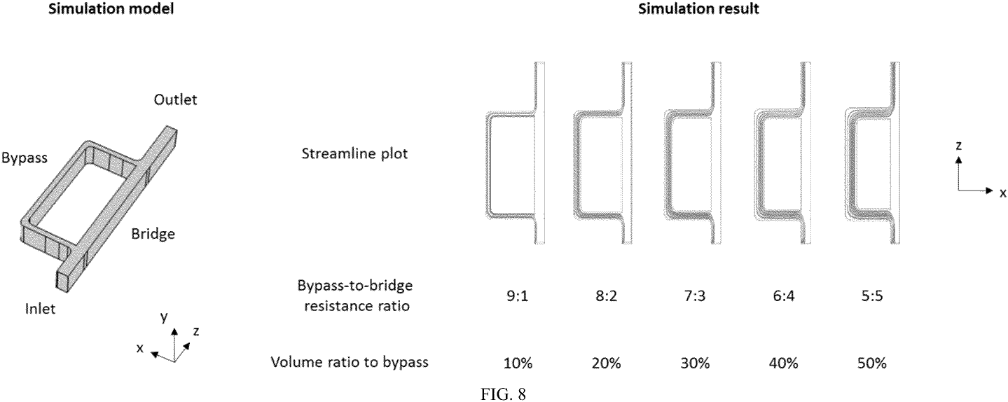

Referring to FIG. 8, a computational fluid dynamic (CFD) simulation of one embodiment of a TIS system with the basic form shows linear relationship between the amount of fluid being divided to a bypass channel and the hydraulic resistance ratio between bypass channel (block D) and the bridge (Block B), suggesting a precise sheath extraction can be achieved by controlling the hydraulic resistance of the channel.

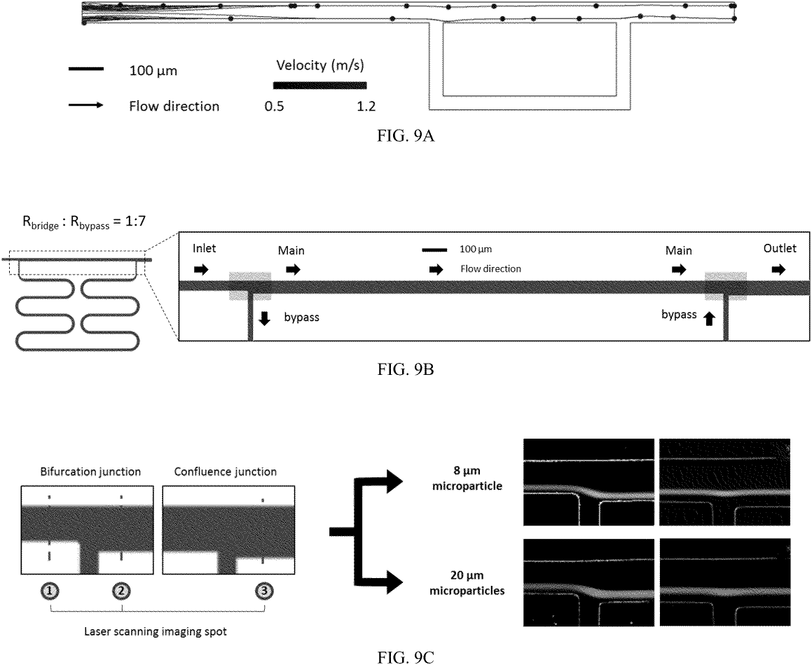

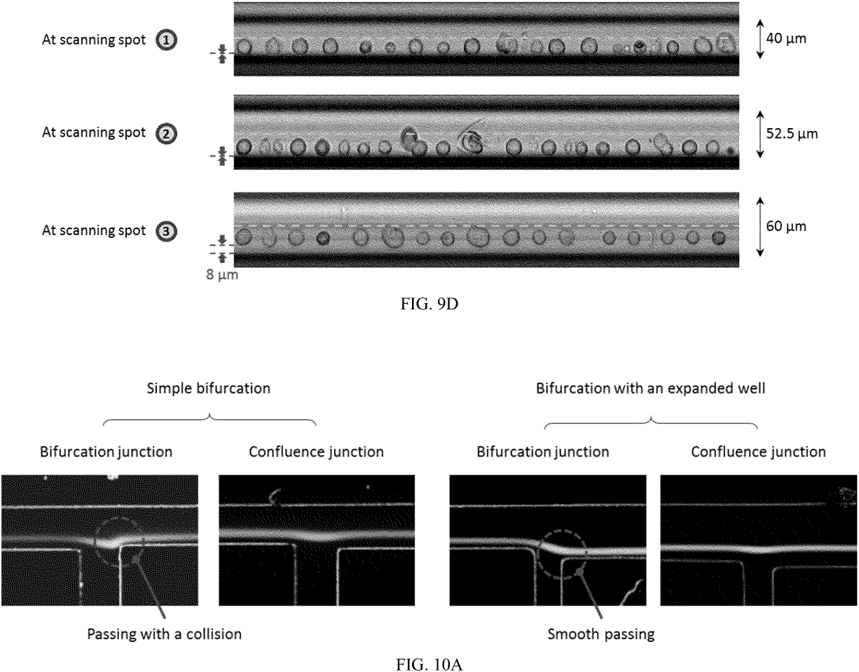

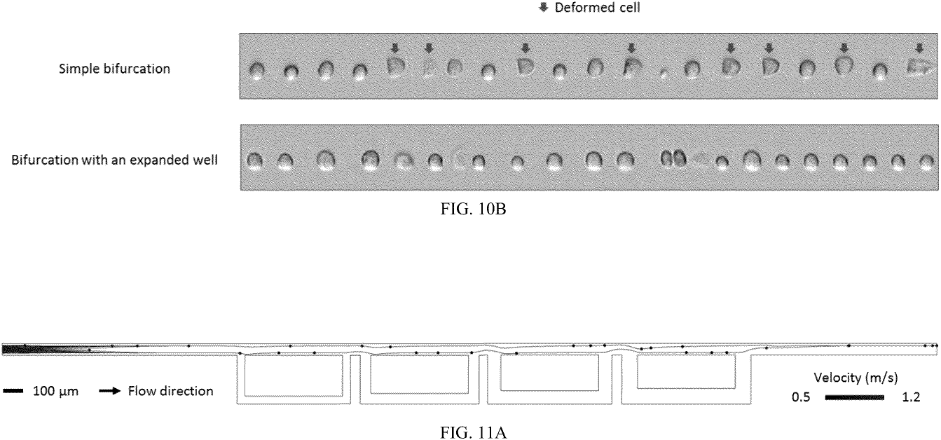

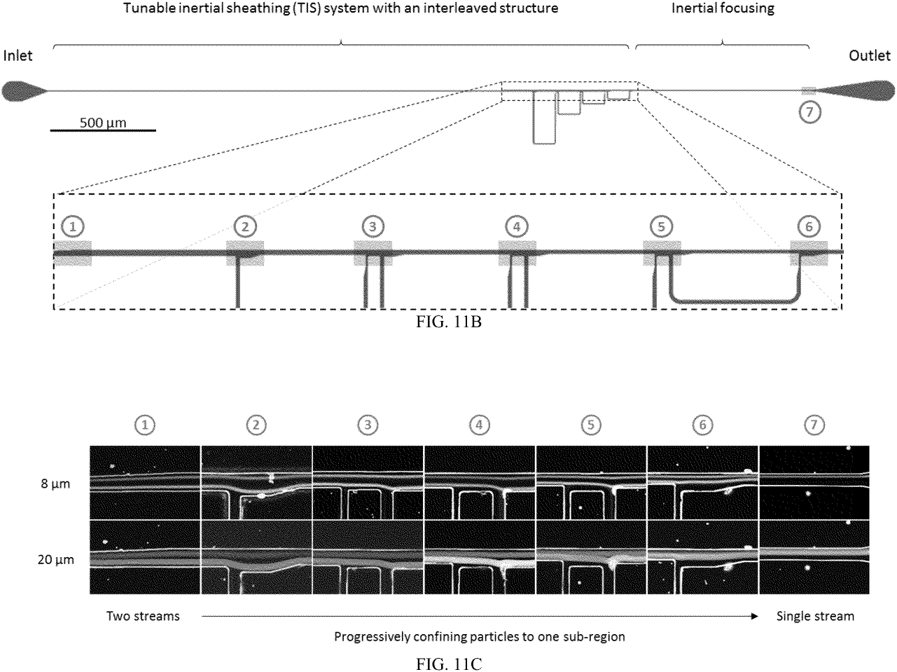

Referring to FIG. 9A, another CFD simulation of one embodiment of a TIS system with the basic form shows the trajectories of particles flowing inside. A temporal upward shift of the bottom stream suggests a successful particle localization by TIS. Now referring to FIG. 9B, in one embodiment, a TIS system with the basic form is shown in scale with a zoom-in view of the block B. The bridge-to-bypass hydraulic resistance ratio is set to be 1 to 7 in this embodiment to generate the sheath fluid with a thickness of one eighth of the channel width. The bypass channel is formed to have a serpentine pattern to illustrate that only the channel resistance affects the amount of the sheath fluid in the branch.

To examine the particles stream shifting, two solutions are injected into the TIS system, each having fluorescent polystyrene beads with a certain size. For example, one solution may have fluorescent polystyrene beads with a diameter of 8 µm and the other solution may have fluorescent polystyrene beads with a diameter of 20 µm. The trajectories of the moving fluorescent polystyrene beads are subsequently recorded.

First, the particles are focused into a single stream in the upstream focusing unit for better visualization and its single trajectory can be viewed in streak images of FIG. 9C. The stream remains in close contact with the wall of the main channel right after passing the bifurcation junction while moving away from the wall of the bridge during the flow through the bridge, indicating that the inertial focusing resets the positions of the particles.

Nevertheless, the stream does not get close to the wall of the main channel after passing the confluence junction, indicating a successful accumulation of the sheath fluid. The same effect is observed when the sizes of the beads vary, suggesting that the accumulation of the sheath fluid can be particle-size insensitive. For better validation, an ultrafast laser scanning microscope is used to image the fast-flowing biological cells, which are more heterogeneous in size distributions, at three different locations as illustrated in FIG. 9C.

Referring to FIG. 9D, cells of varied sizes are focused to form a single stream near the wall of the trunk, same as the microbeads at the scanning spot one. Due to the light diffraction, the wall of the main channel which is the longitudinal side of the channel cross-section after imaging, spans across the focal plane of the microscope. Thus, a part of the wall is out of focus, resulting in a dilated wall with a decreased contrast in FIG. 9D. Moreover, there is no gap formed between the cells and the wall of the main channel as indicated by the red arrow at the scanning spot one of FIG. 9D. In addition, it has been shown that no gap is formed at scanning spot two. However, a 7.5 µm thick gap is formed at the scanning spot three which is approximately one eighth of the channel width of 60 µm. Thus, the results prove that the cross-section partitioning can be precisely achieved by configuring designs of the channel structure for bifurcation and unification of the particle streams.

In addition to the single-stream inertial focusing, the TIS system and methods can also be utilized in the field of deformability cytometry to assess mechanical properties of particles.

Referring to FIG. 10A, in one embodiment, the TIS system is formed without an expanded well that sets the bifurcation boundary to be on the particle single-stream, forcing particles to collide with the channel at the bifurcation junction. Such impacts introduce a significant force to deform the shape of elastic particles into a triangular-like shape. Under a well control, the elasticity of cells can then be quantified by various imaging techniques. On the other hand, an expanded well effectively inhibits the collision while does not affect the performance of sheath accumulation at shown in the confluence junction.

FIG. 10B shows images of cells captured by ultrafast laser scanning microscope before and after the particle collisions. The triangle-like shaped cells indicated by the red arrows, which are significantly deformed cells, are found only after the particle collisions. Therefore, the application of TIS system and methods is not limited to single-stream focusing, but may include a wide spectrum of microfluidic operations requiring precise single-particle positioning.

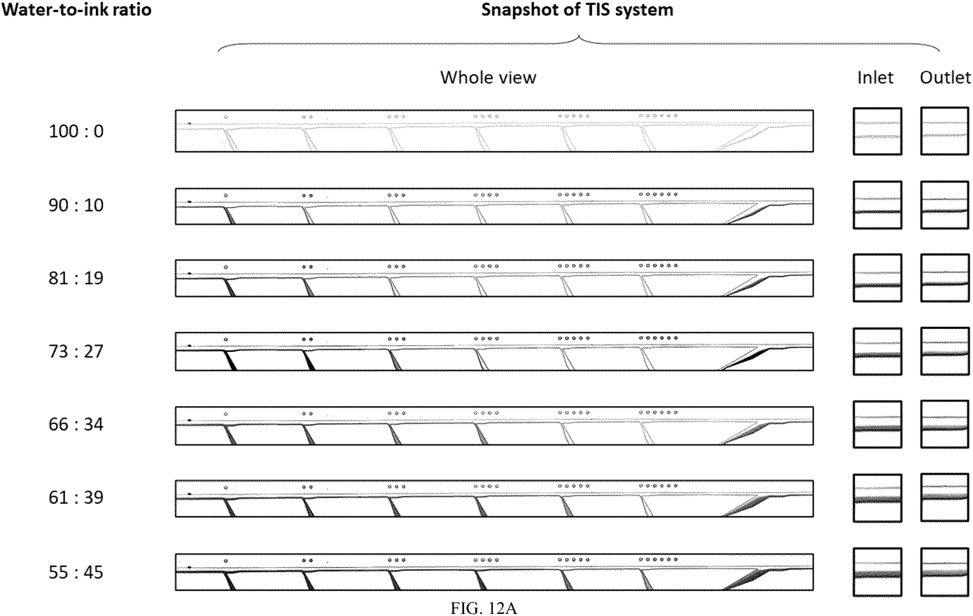

Referring to FIG. 11A, a CFD simulation of one embodiment of a TIS system with the interleaved form shows the trajectories of particles flowing inside. The bottom stream has temporal upward shifts at the confluence junctions with an increasing amplitude and merges with the top stream in the end, suggesting a successful particle localization by a TIS system with the interleaved form.

Referring to FIG. 11B, a microfluidic channel that includes an interleaved form TIS system with a long high-aspect-ratio cuboid channel attached at the downstream is demonstrated by an in-scale schematic representation.

By fixing the widths of bypass channels, the resistance of each bypass channel scales only with the length of the corresponding branch. As a result, the progressively reduced resistance (equivalently progressively increased sheath fluid volume) in the bypass channel is achieved by the staircase-like structure of the TIS system.

Referring to FIG. 11B again, the zoom-in window shows all bifurcation junctions and confluence junctions at which fluorescent streak images are taken. Deionized water containing fluorescent polystyrene beads of a diameter of 8 µm and a diameter of 20 µm are respectively injected into the microfluidic chip. Then, the images of formation and change of the streams are captured and shown in FIG. 11C. Two parallel fluorescence streams are formed after passing the extended high-aspect-ratio cuboid channel due to the inertial focusing as illustrated in Figure 11C(1). Next, the streams are skewed to greater and greater degrees while they travel toward the downstream after each basic form TIS system as shown in the transition regions between the basic forms in FIG. 11C(3) - 11C(5) and finally biased to the upper half after passing through four basic forms as shown in FIG. 11C(6). Then, the two streams, one with the 8 µm beads and the other with the 20 µm beads, are merged into one stream after passing through the second extended high-aspect-ratio cuboid channel as shown in FIG. 11C(7).

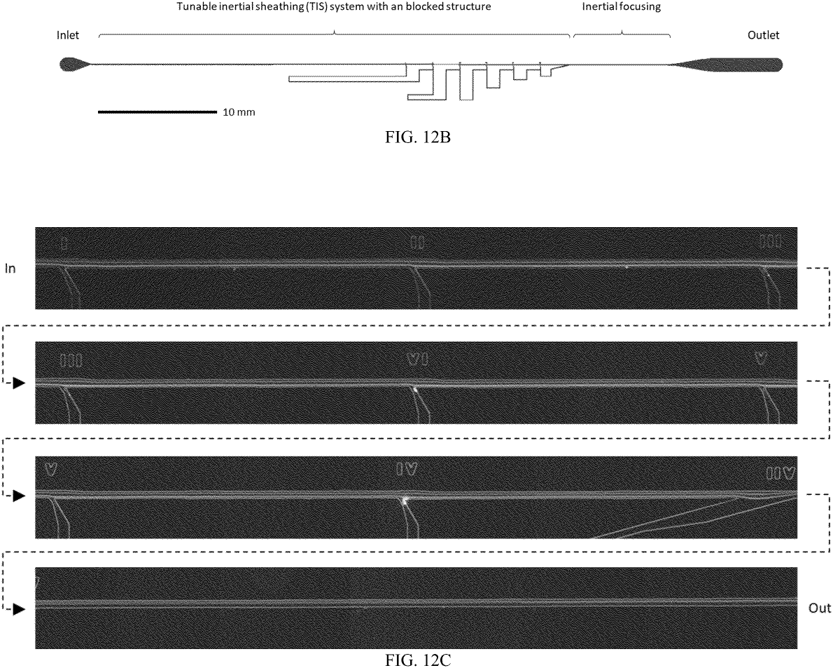

Referring to FIG. 12A, a series of images of co-flow of water and ink in one embodiment of a TIS system with the interleaved form shows the fluid intake by the bypass channel at different ratios of water to ink. The result shows how the bypass channel continuously intakes peripheral fluid from the main channel up to 45% of the total fluid and then returns it back to the main channel.

Referring to FIG. 12B, a microfluidic channel that includes a blocked form TIS system with a long high-aspect-ratio cuboid channel attached downstream is demonstrated by an in-scale schematic representation.

Referring to FIG. 12C, deionized water containing fluorescent polystyrene beads of a diameter of 6 µm is injected into the microfluidic chip. Then, the images of formation and change of the streams across the entire main channel are captured and shown in FIG. 12C. Two parallel fluorescence streams are formed after passing the block A with a high-aspect-ratio cuboid due to the inertial focusing as illustrated. Next, the streams are skewed and recovered while they travel toward the downstream after each block B form of the TIS system and finally biased to the upper half after passing through the confluence junction. Then, the two streams with the 6 µm beads are merged into one stream after passing through the long high-aspect-ratio cuboid channel to complete a single-stream focusing.

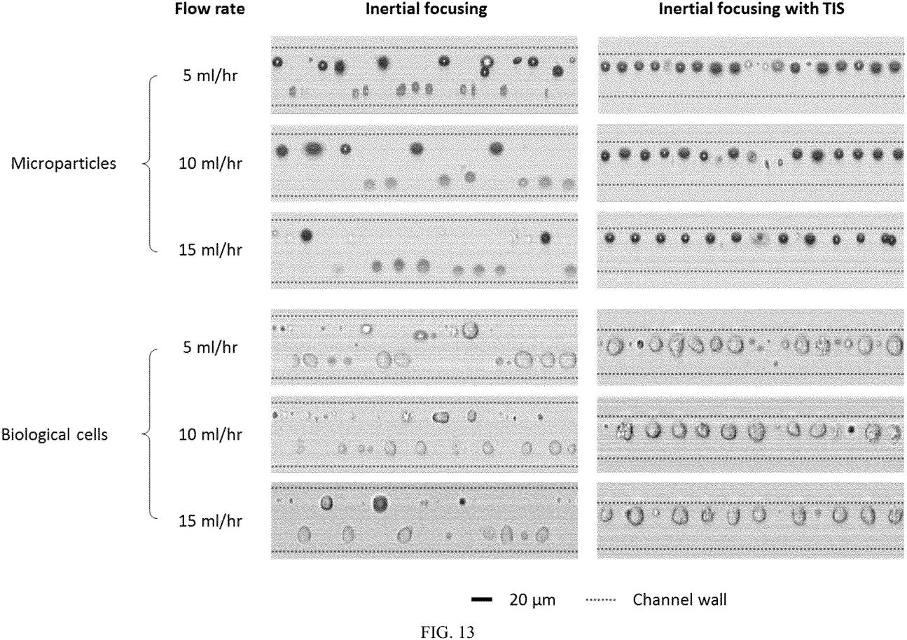

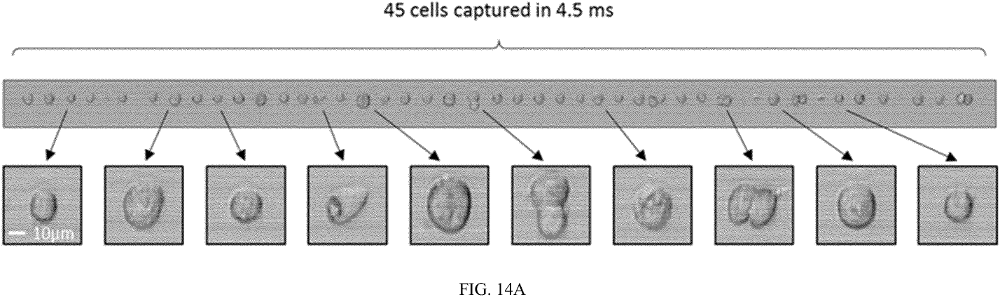

Now referring to FIG. 13, laser scanning images are captured to examine the single stream focusing performance of the TIS assisted inertial focusing when microparticles and biological cells flow through the microfluidic chip at different flow rates. At all flow rates, the laser scanning images captured before the TIS system show two distinctive particle trains, while these images captured after the TIS system show just one particle train in the upper half. In addition, these particles are all “in-focus” which means that the particles are all located within a narrow range of a vertical position in the channel’s cross-sectional area, indicating that a precise 3D focusing is obtained by the TIS system and methods. Moreover, the wide range of applicable flow rates suggests that single-stream inertial focusing given by the TIS system and methods have great tolerance for flow rate variations.

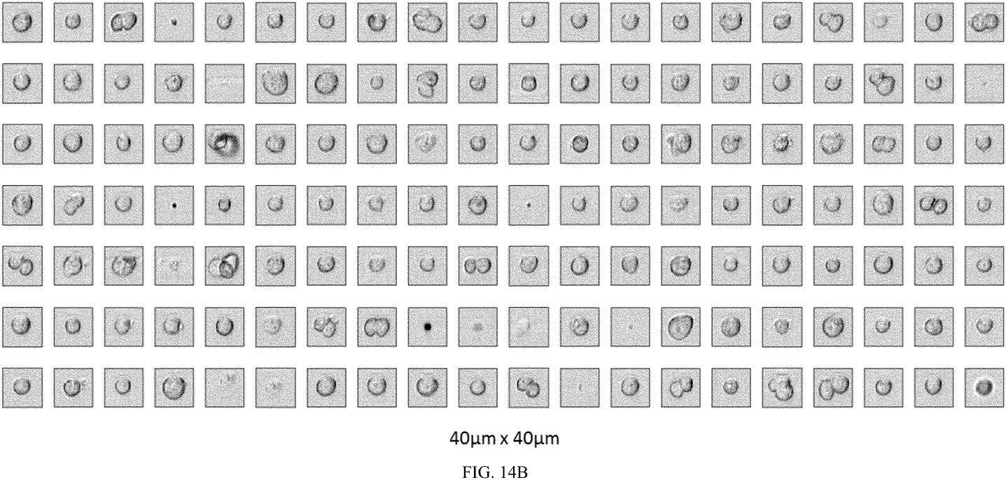

The 3D focusing performance of the TIS system and methods is also tested in high-throughput operations as illustrated in FIGS. 14A and 14B.

Referring to FIG. 14A, an image with compression is shown for 10,000 cells taken within 1 second at the outlet of the microfluidic channel, corresponding to high throughput of 10,000 particles per second. The zoom-in window of FIG. 14A shows an in-scale image of 45 cells. These cells are heterogeneous both in size and in shape but all in sharp focus under the high throughput, demonstrating the advantageous capability of size-insensitive high-throughput 3D focusing of the CPC system and methods. The images of the first 120 cells are shown in FIG. 14B to demonstrate that there is no cheery-picking in the tests for validating the focusing performance. Therefore, an immediate application of the subject invention is particle focusing operations of imaging/non-imaging flow cytometers for high-throughput particle analysis.

The TIS system and methods of the subject invention is advantageous in that they do not require any external sheath fluids or external force fields, thereby inhibiting dilution of the particles to benefit the downstream particle analysis, reducing hydraulic pressure to improve the system robustness, and reducing the manufacturing and operating costs.

Moreover, since the TIS system and methods achieve the single stream focusing based on inertial focusing and particle distribution confinement in contrast to the conventional systems and methods that require the aid of external force field such as secondary/Dean force, the TIS system and methods can be developed without requirement for a subtle balance between inertial lift and secondary forces which complicatedly depends on the flow rate, square of the particle size, and cubic of channel cross-sectional area. Hence, the TIS system and methods have a large tolerance on fluid flow rate control and scalable throughput, cover a broad range of focusable particle sizes, have a large tolerance on manufacturing precision such as photolithography resolution, and simplify the channel design process.

The TIS system and methods of the subject invention are also advantageous in that all particles are focused on the same horizontal plane in co-planar focusing, regardless of size differences of the particles, a feature that is essential for technologies that are sensitive to the variation of particle position in the vertical direction, such as optical analytic technologies, to acquire high-quality data. In particular, such system enables high-throughput imaging flow cytometry with > 10,000 cell throughput by integrating with the high-speed imaging system and camera technology (for example, scientific CMOS), which is employed to capture images of fast flowing cells after the TIS system.

Since the TIS system and methods of the subject invention do not require the use of non-Newtonian fluids including water and biological fluids (for example, phosphate buffer saline and blood) for daily samples, tedious fluid exchange process in the sample preparation is eliminated. Accordingly, not only the operations are simplified and sped up, but also the perturbation introduced to the sample is minimized, enabling analysis of live samples for life science applications.

The TIS system and methods of the subject invention may have widespread applications for various flow cytometry applications and optical interrogation of particles. When a particle sorter is incorporated into the downstream of the TIS system, further analysis and more advanced applications may be achieved. When simulations of the force field and the corresponding particle migration are utilized, the focusing effects can be optimized and enhanced such that sizes of the microfluidic chips designed based on the TIS system and method are minimized.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A microfluidic system for focusing particles suspended in a particle-carrying fluid, comprising:
a microfluidic channel comprising:
a first section configured to perform tunable inertial sheathing (TIS); and
a second section configured to perform inertial focusing.

2. The microfluidic system of claim 1, wherein the TIS internally confines a plurality of particles to have a spatial distribution within one of a plurality of compartments of an inertial force field in a cross-sectional area of the microfluidic channel.

3. The microfluidic system of claim 1, wherein the microfluidic channel is formed to have a cuboid structure.

4. The microfluidic system of claim 2, wherein only one inertial focusing spot exists within the one of the pluralities of compartments of the inertial force field in the cross-sectional area of the microfluidic channel.

5. A tunable inertial sheathing (TIS) system for performing particle localization on a plurality of particles, the TIS system comprising:
a fluidic channel comprising a main channel and a bypass channel;
the main channel comprising:
at least three straight segments;
at least one bifurcating junction; and
at least one confluence junction; and
the bypass channel comprising:
at least one inlet; and
at least one outlet.

6. The TIS system of claim 5, wherein the fluidic channel is made of cyclic olefin copolymer (COC), polymethylmethacrylate (PMMA), or polycarbonate (PC).

7. The TIS system of claim 5, wherein each of the bifurcation and confluence junction has a Y-cross structure, the Y-cross structure comprising:
three ends comprising;
two ends connected to the straight segments; and
one end connected to the bypass channel; and
an expanded well.

8. The TIS system of claim 5, wherein the fluidic channel is formed with a high-aspect-ratio rectangular channel cross section to confine the particles into a pair of focal points in a mid-plane by inertial focusing.

9. The TIS system of claim 5, wherein the fluidic channel has an aspect ratio larger than unity.

10. The TIS system of claim 5, wherein the fluidic channel is made of silicone comprising polydimethylsiloxane.

11. The TIS system of claim 7, wherein the fluidic channel is configured to have a basic form, comprising:
two straight segments for inertial focusing to generate peripheral particle-free fluid;
one bifurcation junction for partitioning the particle-free fluid to the bypass channel from particle-carrying fluid and concentrating the particle-carrying fluid in the main channel; and
one confluence junction for sheathing the particle-carrying fluid by the particle-free fluid in the bypass channel and temporally localize particle distribution within a smaller area of the cross-sectional area of the fluidic channel.

12. The TIS system of claim 11, wherein a slide of particle-free fluid is formed in the bifurcating structure as a self-generated sheath fluid without affecting the inertial focusing.

13. The TIS system of claim 12, wherein a thickness of the particle-free fluid is determined by a Reynolds number of the particle-carrying fluid, sizes of the particles, and geometry of the channel.

14. The TIS system of claim 11, wherein a volumetric flow rate of the particle-free fluid is about equal to or larger than a volumetric flow rate of the particle-carrying fluid in a last straight segment connected to a last confluence junction.

15. The TIS system of claim 5, wherein the TIS system is configured to attain different sheath-extraction conditions, including:
a small-volume extraction;
a large-volume extraction; or
multiple small-volume extractions.

16. The TIS system of claim 15, wherein the small-volume extraction is configured to have a slide of fluid that has a thickness smaller than a distance between a center of a smallest particle to be focused and a nearest wall of the channel.

17. The TIS system of claim 15, wherein the large-volume extraction is configured to have a slide of particle-free fluid which has a thickness smaller than a distance between a center of a largest particle to be focused and a nearest wall and larger than a distance between a center of a smallest particle to be focused and the nearest wall.

18. The TIS system of claim 15, wherein the multiple small-volume extractions are configured to have multiple stages of the small-volume extraction, the large-volume extraction, or a combination of both.

19. The TIS system of claim 15, wherein the fluidic channel is configured to have a pattern, including:
an interleaved form;
a blocked form; or
a mixed form having combinations of the interleaved form, or the blocked form.

20. The TIS system of claim 5, wherein the fluidic channel is configured to have multiple bifurcation and confluence junctions forming an overall asymmetric structure to achieve a volumetric flow rate of the particle-free fluid that is about equal to or larger than a volumetric flow rate of the particle-carrying fluid in a last straight segment connected to a last confluence junction for size-insensitive single-stream particle focusing.

21. The TIS system of claim 5, wherein the fluidic channel is configured to have a varied form to introduce a particle collision in the bifurcation junction for cell deformation.

22. A system for imaging and analyzing a plurality of biological cells, comprising:
the microfluidic system according to claim 1 to focus the biological cells into a single stream;
a microfluidic channel comprising a third section configured to perform second TIS for high-quality optical interrogation of the focused biological cells; and
a real-time image acquisition system for imaging optically in-focused biological cells.

23. The system of claim 22, wherein the optically in-focused biological cells are located within a depth of field of the real-time image acquisition system.

24. The system of claim 22, wherein the image acquisition system records in-focused biological cell image contrasts including:
bright-field contrast;
quantitative phase contrast; and
fluorescence contrast.

25. The TIS system of claim 13, wherein the Reynolds number of the particle-carrying fluid is between 1 and 2000.

26. The TIS system of claim 5, wherein the fluidic channel is formed with a rectangular channel cross section having a height H and a width W each between 1 micrometer and 10 millimeters and an aspect ratio AR between 0 and 0.75 or larger than 1.33 for partitioning inertial force field into two compartments.

27. The TIS system of claim 26, wherein the height H is about 80 micrometers, the width W is between 20 and 60 millimeters, and the aspect ratio AR is between 1.33 and 4.

28. The TIS system of claim 5, wherein the particle size is between 0.1 micrometers and 10 millimeters.

29. The TIS system of claim 5, wherein the particle size is between 5.6 and 30 micrometers.

30. The TIS system of claim 5, wherein the fluidic channel comprises four blocks including a block A that is a straight segment at beginning to initiate inertial focusing to peripherally sheath particles, a block B that is a bifurcation junction dividing certain previously generated sheath fluid to a bypass channel and a straight segment with sufficiently long length recovers the sheathing, a block C that is a confluence junction returning certain sheath fluid to a main channel to temporally localize particles in the straight segment, and a block D that is the bypass channel in which only sheath fluid flows.

31. The TIS system of claim 30, wherein a length L_{A1} of the block A is configured to be on a scale of tens of millimeters and a length L_{B2} of the block B is configured to be on a scale of a few millimeters.

32. The TIS system of claim 30, wherein a length L_{B1} of the block B, a length L_{C1} and a length L_{C2} of the block C each is configured to be on a scale of hundreds of micrometers. 33.
The TIS system of claim 30, wherein a width of a fully developed stream to the block D, W_{FD}, is configured to be between 0 to 60 micrometers.

34. The TIS system of claim 30, wherein a width W_{B2} of the block B and a width W_{C2} each is between 1 micrometer and 10 millimeters, and an angle θ_{B2} of the block B and an angle θ_{C2} of the block C each is between 120 degrees and 180 degrees.

35. The TIS system of claim 30, wherein a width W_{B2} of the block B and a width W_{C2} of the block C each is between 40 micrometers and 100 millimeters, and an angle θ_{B2} of the block B and an angle θ_{C2} of the block C each is between 120 degrees and 170 degrees.
